(19) United States
(12) Patent Application Publication
(10) Pub. No.: US 2016/0002272 A1
(43) Pub. Date: Jan. 7, 2016

(54) SURFACE FUNCTIONALIZED POROUS SILICON MATERIAL AND METHOD OF MAKING THEREOF

(71) Applicants: SPINNAKER BIOSCENCES, INC., Solana Beach, CA (US); THE REGENTS OF THE UNIVERSITY OF CALIFORNIA, Oakland, CA (US)

(72) Inventors: Chia-Chen WU, Taichung City (TW); Michael J. SAILOR, La Jolla, CA (US); Michelle Y. CHEN, Newport Coast, CA (US)

(73) Assignees: THE REGENTS OF THE UNIVERSITY OF CALIFORNIA, Oakland, CA (US); SPINNAKER BIOSCIENCES, INC., Solana Beach, CA (US)

(21) Appl. No.: 14/770,191

(22) PCT Filed: Feb. 25, 2014
(86) PCT No.: PCT/US14/18244

§ 371 Date: Aug. 25, 2015

Related U.S. Application Data
(60) Provisional application No. 61/769,052, filed on Feb. 25, 2013.

(51) Int. Cl. C07F 7/21; A61K 47/24; A61K 9/00

(52) U.S. Cl. CPC C07F 7/21; A61K 9/0051; A61K 47/24

(57) ABSTRACT

The present invention relates generally to a surface functionalized porous containing material and method of making thereof.

### CROSS-REFERENCE TO RELATED APPLICATION

This application claims the benefit of U.S. Provisional Application No. 61/769,052, filed on Feb. 25, 2013, the disclosure of which is incorporated herein by reference in its entirety.

### GOVERNMENT SUPPORT

This work was supported in part by the U.S. National Science Foundation under Grant No. DMR-1210417. The government has certain rights in the invention.

### FIELD OF THE INVENTION

The present invention relates generally to a surface functionalized porous silicon containing material and method of making thereof.

### INTRODUCTION

One of the longstanding chemical challenges in engineering of nanomaterials is to control the placement of different chemistries in spatially distinct regions on a nanoscale object.

Consequently, there is still a need to provide methods that allow control on the placement of different chemical species in nanostructures, such that novel nanostructures can be produced.

### SUMMARY

The present invention provides methodology for differentially modifying the inner pore walls and the pore openings of silicon containing materials to produce novel structures. The method uses an inert liquid to mask the interior of the porous silicon material, while the exterior surface and the pore mouths are subjected to a chemical reaction with a reactive liquid. The novel resulting terminal surface allows further chemical functionalization to bind ligands of interest.

The present invention provides a silicon containing material having a plurality of pores, the material comprising: a) an exterior surface region comprising a first terminal group; and b) a interior pore surface region comprising a second terminal group, wherein the first terminal group and the second terminal group are different from each other and are chemically linked to the material.

In accordance with certain embodiments, the present invention provides a silicon containing material having a plurality of pores; the material comprising: a) an exterior surface region comprising a first terminal group comprised of hydride, hydrocarbon, carboxylic acid, amine, haloalkane, aromatic hydrocarbon, thiol, peptide, carbon, silicon oxide, silicon dioxide, or mixtures thereof; and b) an interior pore surface region comprising a second terminal group selected from the group consisting of hydride, hydrocarbon, carboxylic acid, amine, haloalkane, aromatic hydrocarbon, thiol, peptide, carbon, silicon oxide, silicon dioxide, or mixtures thereof.

An aspect of the invention provides a method of treating a disease or disorder of the eye comprising injecting into the eye a silicon containing material comprising: a) an exterior surface region comprising a first terminal group; and b) an interior pore surface region comprising a second terminal group, wherein the first terminal group and the second terminal group are different from each other and are chemically linked to the material.

Another aspect of the invention provides a method of preparing a silicon containing material comprising an exterior surface comprising a first terminal group and a pore surface comprising a second terminal group, wherein the first terminal group and the second terminal group are different from each other and are chemically linked to the material, the method comprising: providing the material; contacting the material with an inert liquid to infiltrate the interior pore surface region; an immersing the material in a reactive liquid; wherein the reactive liquid is immiscible or partially immiscible with the inert liquid.

### DESCRIPTION OF THE DRAWINGS

The drawings illustrate generally, by way of example, but not by way of limitation, various embodiments discussed in the present document.

FIG. 1 is a schematic illustration of a process of preparing a porous silicon film according to an embodiment of the present disclosure.

FIG. 2 is a schematic illustration of a process of preparing a porous silicon film according to an embodiment of the present disclosure.

FIG. 3 shows the FTIR spectra of a porous Si film according to an embodiment of the present disclosure at various steps of the process outlined in FIG. 1. FIG. 3 (A) freshly etched porous Si thin film presents hydride species throughout the inner and outer pore surfaces; (B) sample partially oxidized in air for 2 h at 600° C.; (D) after infiltration with octane and exposure to aqueous HF (0.77%) for 600 s; (E) after thermal hydrosilylation with 1-dodecene (sample was rinsed and dried prior to acquisition of the spectrum).

FIG. 4 shows a graph using an optical measurement of the fractional filling of a partially oxidized porous Si—SiO₂ film containing the indicated organic liquids film according to certain embodiments of the present invention, as a function of time exposed to liquid water.

FIG. 5 is a graphical representation showing various EDX spectra obtained from a cross-section of a porous Si film according to certain embodiments of the present invention. (A) EDX spectrum obtained at a distance 1 μm from the top of the modified porous Si film; (B) an EDX spectrum obtained at a distance 1 μm from the bottom of the modified porous Si film (i.e., 1 μm from the interface between the porous Si layer and the bulk silicon substrate).

FIG. 6 is a graphical representation showing various EDX spectra obtained from plan view images of a porous Si film according to certain embodiments of the present invention. (A) 10-bromo-1-decyl-terminated porous Si, and (B) thermally oxidized porous Si sample.

FIG. 7 shows a measured sessile contact angle of an alkyl-modified liquid masked porous Si sample as a function of time of exposure of an inert liquid-masked film to aqueous HF according to an embodiment of the present disclosure.

FIG. 8A shows an optical reflectance spectrum of a Si sample prepared by the process according to an embodiment of the present disclosure. FIG. 8B shows the quantity 2nL measured as a function of time during water infiltration into a control sample consisting of partially oxidized porous Si, without the hydrophobic barrier layer.

FIG. 8C shows the quantity 2nL measured as a function of time during water infiltration into a sample containing dodecyl barrier layer according to an embodiment of the present disclosure.

FIG. 9 is a photograph using a scanning electron microscope which shows a plan-view (A), and a cross-section (B) of a freshly etched porous Si film according to certain embodiments of the present invention. Scale bars are 50 nm (A) and 2 μm (B) respectively.

FIG. 10 illustrates the selective chemical modification of the pore mouths in porous Si according to an embodiment of the present disclosure that allows controlled transport/release of molecular payloads.

FIG. 11 illustrates release profiles of rhodamine B into aqueous PBS buffer from partially oxidized porous Si layers containing different top barrier layers: () porous Si/SiO₂ layer with no barrier layer (sample not subjected to liquid masking procedure); (▪) porous Si/SiO₂ layer subjected to liquid masking procedure, with dodecyl-terminated top layer displaying a water contact angle of 80°; (♦) porous Si/SiO₂ layer subjected to liquid masking procedure, with dodecyl-terminated top layer displaying a water contact angle of 118°. CA stands for water contact angle, measured on surface of the modified (or non-modified) porous porous Si/SiO₂ layer. Each data point in the curves was averaged from three samples and the error bars indicate standard deviation.

### DETAILED DESCRIPTION

As used herein, the term “silicon containing material” refers to any material including at least one silicon atom per formula unit, or at least 0.1 percent silicon by mass. Examples of least one silicon atom per formula unit, or at least 0.1 percent silicon by mass include silicon (including crystalline and polycrystalline silicon), polysiloxanes, silanes, silicones, and siloxanes. Examples include SiO₂, Si, aminopropyldimethyl siloxane, and tetramethoxysilane. The term “silicon containing material” is interchangeable with the term “material,” which refers to the material of the present invention obtained at any stage of the process of making.

The term “region” refers to an area or a portion of a surface of the present embodiments.

The present invention provides a silicon containing material having a plurality of pores, the material comprising an exterior surface region comprising a first terminal group, and an interior pore surface region comprising a second terminal group, where both the first terminal and second terminal groups are chemically linked to the material and are different from each other.

The material of the present invention contains a plurality of pores with an exterior surface region (i.e., outer surface of the pore) and an interior pore surface region (i.e., inner surface of the pore walls). These surface regions may extend several hundred nanometers into the material. The exterior surface region and the interior pore surface region each include a chemical functional group, which are different from each other, namely a first terminal group and a second terminal group, respectively.

In certain embodiments, one of the first and second terminal groups of the silicon containing material may include a hydride terminal group, such as, silicon hydride. In certain embodiments, at least one of the hydride terminal groups may be modified to an organosilane. Such organosilanes may include an alkyl, a carboxylic acid, an ester, an amine, a protein, an oligonucleotide, a short chain peptide, a sugar, a polysaccharide, a fatty acid, or mixtures thereof. In certain embodiments, the organosilane may include an alkyl. Such an alkyl group may be linear or branched. In further embodiments, the alkyl may contain other chemical substituents, such as a halogen. In further embodiments, the organosilane may include an alkyl having between about 1 and about 30 carbon atoms, having between about 5 and about 20 carbon atoms, or having between about 8 and about 14 carbon atoms. That is, the silicon hydride surface material may be converted (in part) to a silicon-alkyl surface material, in which a hydrocarbon is grafted to the silicon surface via Si—C bonds.

In certain embodiments, the other one of the first and second terminal groups comprises carbon, silicon oxide, silicon dioxide, titanium oxide, iron oxide, aluminum oxide, or mixtures thereof. In one embodiment, the other one of the first and second terminal groups comprises silicon oxide. In certain embodiments, the other one of the first and second terminal groups comprises carbon, where the carbon can be prepared by pyrolysis of carbon-containing polymers, as described in Kelly, T. L.; Gao, T.; Sailor, M. J., Carbon and Carbon/Silicon Composites Templated in Microporous Silicon Rugate Filters for the Adsorption and Detection of Organic Vapors. Adv. Mater. 2011, 23, 1776-1781.

In one embodiment, the present invention provides a material having a first terminal group including a hydride terminal group, and the second terminal group including silicon oxide, silicon dioxide, or mixtures thereof. In another embodiment, the present invention provides a material having a first terminal group of material including silicon oxide, silicon dioxide, or mixtures thereof, and the second terminal group including a hydride terminal group. In further embodiments, the hydride terminal groups are modified to an organosilane as disclosed herein.

The first or second terminal group including silicon oxide, silicon dioxide, or mixtures thereof may be further modified to include hydrocarbon, carboxylic acid, amine, haloalkane, aromatic hydrocarbon, thiol, peptide, carbon, or mixtures thereof. These modified terminal groups may be bonded to the surface of the material by bonds to silicon, or bonds to silicon oxide or silicon dioxide.

The silicon containing material of the present invention may be in the form of a film or a particle. The thickness of the film generally ranges from about 5 nm to 500 microns, from about 50 nm to 100 microns, or from about 1 microns to 20 microns. In certain embodiments, the material is a particle. The diameter of the particle generally ranges from about 10 nm to about 300 microns, from about 10 nm to about 100 microns, or from about 2 nm to about 50 nm.

The material of the present invention may be microporous or mesoporous silicon. The material may have a porous structure with an open porosity from about 5% to about 95% based on the total volume of the material. In further embodiments, the material may have an open porosity from about 20% to about 80%, or from about 40% to about 70% based on the total volume of the material. The average pore diameter of the porous silicon material of the present invention is from about 1 nm to about 300 nm, from about 1 nm to about 80 nm, or from about 10 nm to about 50 nm.

A substance, such as a drug or a non-drug, may be loaded into at least one of the pores of the material of the present invention. Examples of a drug substance include, but are not limited to, a small molecule, a protein, a peptide, an oligonucleotide, a nucleic acid, and mixtures thereof. Non-limiting examples of protein-based drug formulations include LUCENTIS® (ranibizumab), AVASTIN® (bevacizumab), and aflibercept (EYLEA®, or VEGF trap-eye®). Non-limiting examples of small molecule-based drug formulations include Foscarnet, doxorubicin, daunorubicin, and rapamycin. Non-limiting examples of oligonucleotide-based drug formulations include GS-101 antisense oligonucleotide, anti-vascular endothelial growth factor (VEGF) oligonucleotide, complementary micro RNA, small interfering RNA (siRNA, or short interfering RNA or silencing RNA). Examples of a non-drug substance include, but are not limited to, an organic dye, an inorganic complex, a metal, a metal oxide nanoparticle, and mixtures thereof.

The drug or the non-drug substance may be attached to the interior pore surface region of the material of the present invention. The interior pore and exterior surface regions of the material can be chemically or physically configured to affect the rate of transport or release of the drug or the non-drug substance.

Certain embodiments of the invention provide a method of treating a disease or disorder of the eye comprising injecting into the eye a silicon containing material of the present invention. Particularly, the present invention provides a method of treating intraocular diseases, such as age-related macular degeneration (ARMD), choroidal neovascularization (CNV), uveitis, diabetic retinopathy, retinovasclar disease, retinal detachment (PVR), and glaucoma.

Certain embodiments of the invention provide a method of preparing the silicon containing material of the present invention. The method includes providing a silicon containing material (porous silicon/porous Si) comprising an exterior surface comprising a first terminal group and an interior pore surface comprising a second terminal group;

contacting the material with an inert liquid to infiltrate the interior pore surface; and immersing the material in a reactive liquid. The porous silicon can be prepared by electrochemical etch of silicon. The porous silicon can also be prepared by chemical (so-called stain) etch of silicon. The porous silicon can also be prepared by chemical reduction of silicon oxide or silicon dioxide. The method modifies the interior pore surface of the pore walls and the pore openings (i.e., the exterior surface) of the material of the present invention. The method employs two immiscible liquids: an inert liquid and a reactive liquid. Generally, the inert liquid can be used as a chemical resist. The inert liquid can be infiltrated into the pores to mask the interior of the porous material of the present invention, while the exterior surface and the pore mouths of the material are subjected to a chemical reaction with a reactive liquid.

The inert liquid may be a hydrophobic organic liquid, for example, alkane, haloalkane, benzene derivative, fatty alcohol, and mixtures thereof. In certain embodiments, the inert liquid includes a C₄-C₁₂ alkane. Examples of suitable inert liquid include, but are not limited to, butane, pentane, hexane, heptane, octane, nonane, decane, dodecane, butanol, pentanol, hexanol, silicone oil, heptanol, and octanol, to aromatics, such as benzene, ethyl benzene, toluene, xylenes, and mixtures thereof.

The reactive liquid may be hydrofluoric acid (HF), an oxidizing agent, or mixtures thereof.

In certain embodiments, the material of the present invention can be oxidized prior to the step of contacting the material with an inert liquid. In certain embodiments, the material can be oxidized thermally, for example at a temperature of from about 300° C. to about 1000° C., at a temperature of from about 400° C. to about 800° C., at a temperature of from about 500° C. to about 700° C. In certain embodiments, both the exterior and the pore surfaces of the material are oxidized to remove the hydride terminal group.

In other embodiments, the material of the present invention can be oxidized following the step of contacting the material with an inert liquid. In certain embodiments, the material can be oxidized by immersing the porous material with the inert liquid infiltrated in the pore surface in hydrogen peroxide. In certain embodiments, the exterior surface of the material is oxidized to remove the hydride terminal group to form the first terminal group.

In certain embodiments, the method of the present invention further includes the step of heating the material with a hydrosilylation agent (i.e., hydrosilylation reaction). The hydrosilylation reaction includes contacting the material with a hydrosilylation agent having from 1 to 30 carbon atoms, from 4 to 20 carbon atoms, from 8 to 14 carbon atoms containing at least one unsaturated hydrocarbon group (e.g., —CH═CH₂ or —C≡CH), for example, an alkene, an alkyne, and mixtures thereof. The unsaturated hydrocarbon group may be at the terminal end of the hydrosilylation agent or in the interior portion of the hydrosilylation agent. Examples of a hydrosilylation agent in which the unsaturated hydrocarbon group is at the terminal end include 1-octene or 1-dodecene. Examples of a hydrosilylation agent in which the unsaturated hydrocarbon group is at the interior portion include 2-octene or 4-dodecene. Such hydrosilylation agents may include a functional group, but are not limited to, carboxylic acid, ester amine, and mixtures thereof. Suitable hydrosilylation agents include, but are not limited to, 1-dodecene, 10-bromo-1-decene, 1-octene, 1-decene, 2-octene, 4-dodecene, undecylenic acid, 10-undecenoic acid, and 10-ethyl undecenoate.

Typically, the ranges of hydrosilylation reaction temperature can be from 50° C. to 300° C., from 80° C. to 250° C., or from 80° C. to 150° C., and the ranges of hydrosilylation reaction time can be from 1 minutes to 3 hours, from 10 minutes to 2 hours, or from 10 minutes to 1 hour.

In certain embodiments, the organosilane produced from the hydrosilylation reaction includes an alkyl, a carboxylic acid, an ester, an amine, a protein, an oligonucleotide, a short chain peptide, a sugar, a polysaccharide, a fatty acid, or mixtures thereof (these groups are referred to as the R groups of SiRX₃), which occupy a pendant position on an SiRX₃, where X represents neighboring silicon atoms on the porous Si surface.

When one of the exterior and pore surfaces of the material contains a hydride group, the step of heating the material with a hydrosilylation agent may modify the hydride group contained on the surface to an organosilane containing surface. In this process, a surface silicon hydride species having at least one SiH group in the molecule is reacted with the carbon-carbon multiple bonds of the unsaturated (i.e., containing at least one carbon-carbon double or triple bond), optionally in the presence of a hydrosilylation catalyst or visible or ultraviolet light. Suitable hydrosilylation catalysts for use in the present invention include H₂PtCl₆ (Spier's catalyst), or Platinum(0)-1,3-divinyl-1,1,3,3-tetramethyldisiloxane (Kerstedt's catalyst).

In certain embodiments, the hydrosilylation reaction step produces a hydrophobic layer on the surface of the material of the present invention when contacted with the hydrosilylating agent described herein. In one embodiment, a hydrophobic layer is present on the exterior surface while the pore surface is hydrophilic in nature. In another embodiment, a hydrophobic layer is present on the interior pore surface while the exterior surface is hydrophilic in nature. The thickness of the hydrophobic layer can vary between 1 and 10 percent, from 1 to 90 percent, or from 10 to 50 percent of the total thickness of the porous material.

A substance (i.e., drug or non-drug) as described herein can be loaded onto the pore surface of the material of the present invention. The rate of release of the substance can be controlled by the placement of different chemical species/functional groups on the surfaces (i.e., exterior and pore surfaces) of the material. For example, in one embodiment, when the exterior surface contains a hydrophobic terminal group, the release time of the substance across the hydrophobic barrier may be longer than that across a surface without a hydrophobic barrier. Such a hydrophobic layer over a hydrophilic inner pore structure is reminiscent of the structure of a liposome, and selective transport of a molecular species (rhodamine B) across the hydrophobic barrier has been demonstrated. (Ruminski, A. M.; Moore, M. M.; Sailor, M. J., Humidity-Compensating Sensor for Volatile Organic Compounds using Stacked Porous Silicon Photonic Crystals. Adv. Funct. Mater. 2008, 18 (21), 3418-3426; Kilian, K. A.; Böcking, T.; Gaus, K.; Gooding, J. J., Introducing Distinctly Different Chemical Functionalities onto the Internal and External Surfaces of Mesoporous Materials. Angew. Chem., Int. Ed. 2008, 47 (14), 2697-2699).

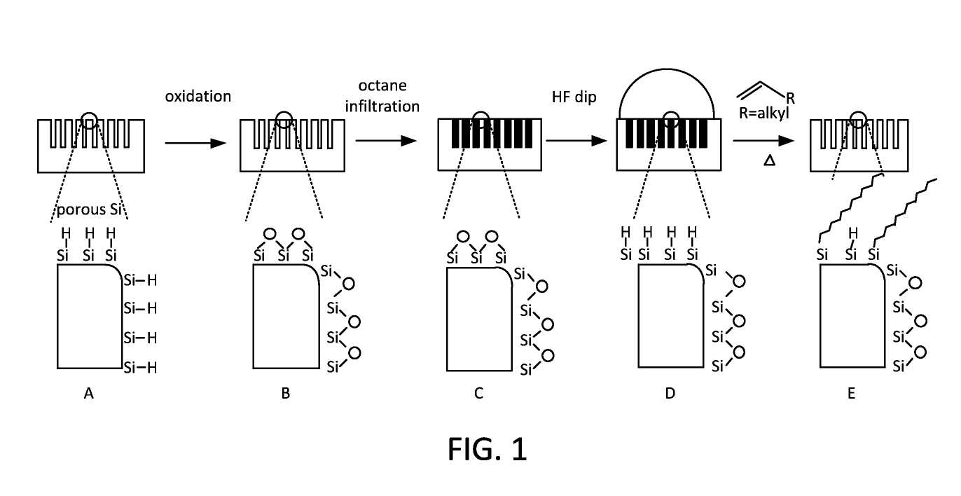

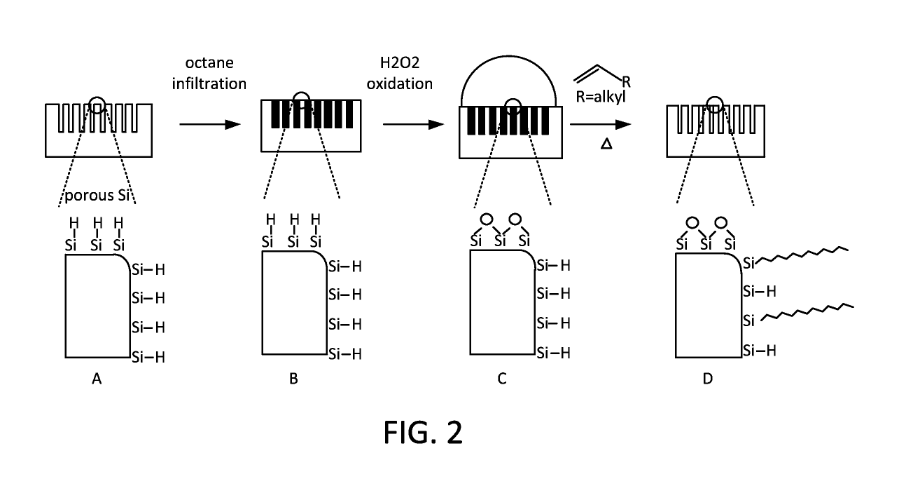

The invention is further described in accordance with certain embodiments, for example, the schematic illustrations shown in FIGS. 1 and 2.

FIG. 1 is one representative schematic illustration of a method of preparing a silicon containing material of the present embodiments. Particularly, the scheme illustrates a selective chemical modification method of preparing a porous Si film: (A) freshly etched porous Si consists of a skeleton of crystalline silicon features with hydride species capping the skeleton surfaces; (B) mild thermal oxidation removes the Si—H species and generates a thin layer of silicon oxide covering the silicon skeleton; (C) the porous Si—SiO₂ layer is then infiltrated with an inert liquid (e.g., octane); (D) immersion of the inert liquid-infiltrated sample in aqueous HF forms an immiscible interface that penetrates into the pores; action of HF on the silicon oxide removes this oxide and places Si—H species on the remaining silicon skeleton; this reaction is self-limiting due to the Si—H surface and the immiscibility of the inert liquid and water; the extent of penetration of the Si—H surface into the porous layer is dependent on the time of exposure to HF; (E) thermal hydrosilylation of the newly generated Si—H surface with an alkene, alkyne, a mixture of alkenes, or a mixture of alkynes selectively adds the alkene, alkyne, mixture of alkenes, or mixture of alkynes across the Si—H bond of the Si—H species, resulting in a spatially resolved surface modification.

Referring to FIG. 1, in certain embodiments, the method of the present invention provides a uniform, hydrophilic silicon oxide in the inner pores and silicon hydride moieties on the opening of the pores. The Si—H and Si—O surfaces can then be modified using subsequent, orthogonal chemical reactions. In one embodiment, the hydrosilylation reaction on the Si—H surface can be performed using an alkane (e.g., dodecene), which yields a hydrophobic layer on the pore mouths. The hydrophobic alkyl species (e.g., dodecyl) at the mouths of the pores may form a barrier for molecular transport, which can decrease the rate of leaching (into water) of a hydrophilic test molecule that is pre-loaded into the sample by several fold, for example, from about 2 to about 20,000 folds, from 2 to about 100 folds, from about 5 to about 20 folds, or from about 7 to about 10 folds.

FIG. 2 is another representative schematic illustration of a method of preparing a silicon containing material of the present embodiments. The scheme illustrates a selective chemical modification method of preparing a porous Si film having an inverse structure relative to that shown in FIG. 1, where the interior wall (or pore surface) is hydrophobic and the exterior is hydrophilic: (A) Freshly etched porous silicon consists of a skeleton of crystalline silicon features with hydride species capping throughout the skeleton surfaces; (B) an inert liquid (e.g., octane) is then infiltrated into the porous silicon layer; (C) immersion of the octane-infiltrated sample in aqueous hydrogen peroxide (H₂O₂) forms an immiscible interface that penetrates into the pores. Action of H₂O₂ at the interface of the two immiscible liquids removes the Si—H species and generates Si—O species; (D) thermal hydrosilylation of the remaining Si—H species at inner pores with an alkene, alkyne, a mixture of alkenes, or a mixture of alkynes selectively adds the alkene, alkyne, mixture of alkenes, or mixture of alkynes across the Si—H bond of the Si—H species, resulting in a spatially resolved surface modification.

Referring to FIG. 2, in certain embodiments, the method of the present invention provides silicon hydride moieties in the inner pores and a uniform, hydrophilic silicon oxide on the opening of the pores. The Si—H and Si—O surfaces can then be modified using subsequent, orthogonal chemical reactions. In one embodiment, the hydrosilylation reaction on the Si—H surface can be performed using an alkene (e.g., dodecene), which yields a hydrophobic layer in the inner pores.

In certain embodiments, the present invention provides a method of preparing a silicon containing material comprising an exterior surface comprising a first terminal group and a pore surface comprising a second terminal group, wherein the first terminal group and the second terminal group are different from each other and are chemically linked to the material, the method comprising: providing a material; thermally oxidizing the material; contacting the material with an inert liquid to infiltrate the pore surface; and immersing the material in a reactive liquid; wherein the reactive liquid is immiscible or partially immiscible with the inert liquid.

In certain embodiments, the present invention provides a method of preparing a silicon containing material comprising an exterior surface comprising a first terminal group and a pore surface comprising a second terminal group, wherein the first terminal group and the second terminal group are different from each other and are chemically linked to the material, the method comprising: providing a material; contacting the material with an inert liquid to infiltrate the pore surface; immersing the material with the inert liquid infiltrated in the pores surface in hydrogen peroxide; and immersing the material in a reactive liquid; wherein the reactive liquid is immiscible or partially immiscible with the inert liquid.

It will be readily apparent to one of ordinary skill in the relevant arts that other suitable modifications and adaptations to the materials and methods described herein may be made without departing from the scope of the invention or any embodiment thereof. Having now described the present invention in detail, the same will be more clearly understood by reference to the following Examples, which are included herewith for purposes of illustration only and are not intended to be limiting of the invention.

### EXAMPLE

### Example 1

Preparation of Porous Si Samples. Mesoporous Si films were prepared by anodic electrochemical etch of highly boron-doped 0.9-1 mΩ-cm resistivity, p-type silicon wafers polished on the (100) crystallographic face (Siltronix, Inc.) in a 3:1 v:v solution of 48% aqueous hydrofluoric acid (ACS grade, Macron Chemicals, Fisher Scientific), and absolute ethanol (Rossville Gold Shield Chemicals) in an electrochemical cell that exposed 1.2 cm² of the wafer to the electrolyte, as previously described. (Sailor, M. J., Porous Silicon in Practice: Preparation, Characterization, and Applications. Wiley-VCH: Weinheim, Germany, 2012; p 249). A constant current density of 15 mA/cm² was applied for 9 min, using a 16 mm diameter ring-shaped Pt wire loop as the counter electrode. Subsequent to etching, samples were rinsed with ethanol and dried under a stream of dry nitrogen.

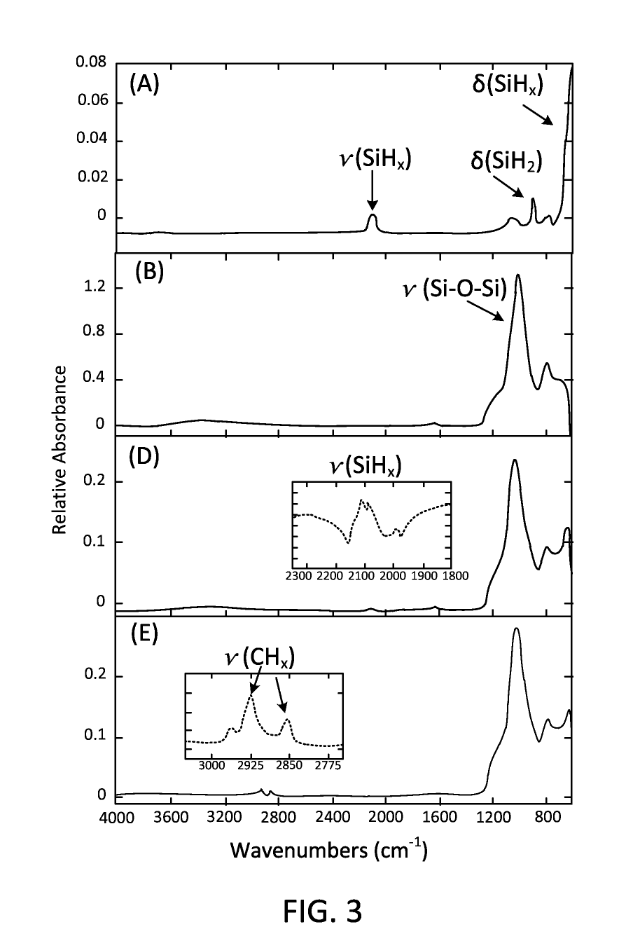

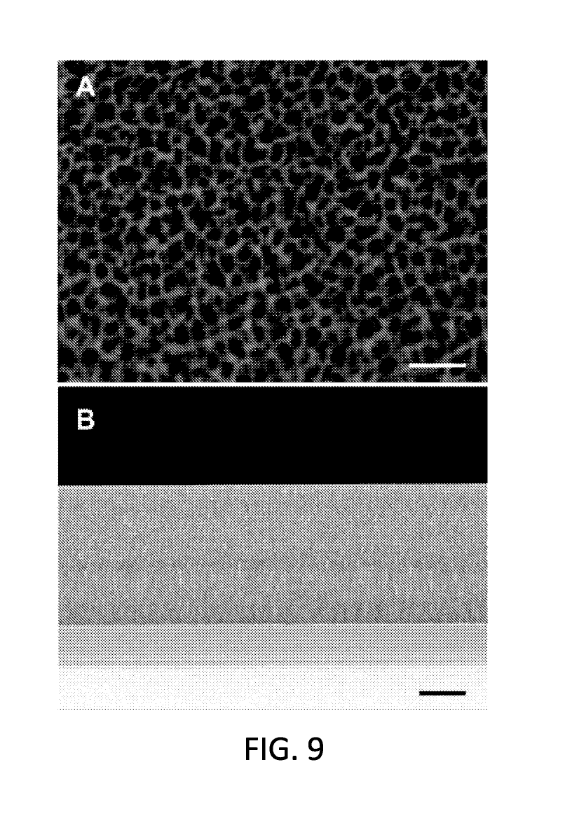

The as-formed (freshly etched) porous Si thin film presents hydride species throughout the inner and outer pore surfaces, as confirmed by Fourier transform infrared (FTIR) spectroscopy (FIG. 3A). Typical porous films consisted of pores of diameter 7±2 nm and film thickness 6.1±0.2 μm (determined by scanning electron microscopy, see FIG. 9), with a total open porosity of 47% (determined by the spectroscopic liquid infiltration method assuming Si skeleton refractive index of 2.48).

### Example 2

Chemical Modification of Porous Si Samples. The method for differential modification of the inner/outer surfaces of a porous Si layer is summarized in FIG. 1.

Thermal oxidation (FIG. 1B). The partially oxidized porous Si—SiO₂ films were prepared by thermal oxidation in a tube furnace (Lindgerg Blue M) at 600° C. for 2 hr in ambient air. This process removed the Si—H species, and yielded a thin layer of silicon oxide coating the entire nanostructured Si skeleton, both inside and outside the pores. The FTIR spectra shown in FIG. 3B illustrates the disappearance of the Si—H bond. The porosity of the film at this point decreased to 38%, which was determined by the spectroscopic liquid infiltration method assuming porous Si—SiO₂ skeleton refractive index of 1.97. The spectroscopic liquid infiltration method is disclosed in Segal, E.; Perelman, L. A.; Cunin, F.; Renzo, F. D.; Devoisselle, J.-M.; Li, Y. Y.; Sailor, M. J., Confinement of Thermoresponsive Hydrogels in Nanostructured Porous Silicon Dioxide Templates. Adv. Funct. Mater. 2007, 17, 1153-1162, which is incorporated herein by reference in its entirety.

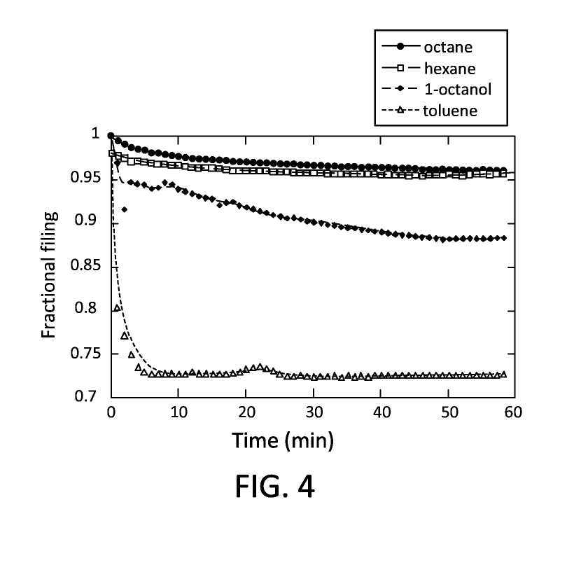

Octane infiltration (FIG. 1C). The film was then mounted in a Teflon cell, and a small quantity of octane was introduced to fill the porous nanostructure. Although the oxidized surface imparts a decidedly hydrophilic nature to the material (sessile contact angle 10±3°), it was found that octane, hexane, 1-octanol, and toluene could penetrate into the porous Si film despite their hydrophobic property. All these liquids were not easily displaced upon immersion in liquid water, although octane showed the best retention behavior as illustrated in the optical measurement studied (See, Example 3). Optical interferometry indicated that less than 3% of the octane in the pores was exchanged by water after 60 min of immersion (See, FIG. 4). Therefore, octane was used as the inert liquid for the subsequent experiments.

HF Dip (FIG. 1D).—Deionized water (2 mL) was added to the cell containing the octane-wetted porous Si film, and the excess octane was observed to float to the surface of the water due to its lower density (0.6986 g/cm³, 25° C.) compared to water (0.9970 g/cm³, 25° C.). An aliquot of 1.55% by volume of aqueous (49%) HF in deionized water (2 mL) was then added to the cell, giving the final HF concentration of 0.77%. The sample was allowed to sit quiescently for 10 min. The FTIR spectrum of a sample removed from the reaction at this point displayed a small signal assigned to Si—H vibrations. The two strongest bands in this region of the spectrum, at 2110 and 2090 cm⁻¹, can be assigned to v_{siH2} and v_{siH} stretching modes, respectively. It is noteworthy that, although the FTIR spectrum displayed a strong silicon oxide band (˜1100 cm⁻¹), the Si—H stretching region of the spectrum showed no evidence of OₓSi—H_{y} species (silicon hydride stretching modes for silicon containing back-bonded oxygen atoms), which are expected to appear in the spectrum at 2160-2260 cm⁻¹. This is indicative of a sharp demarcation between the Si—H and the SiO₂ surface regions of the porous Si—SiO₂ layer. The sessile contact angle measured at this stage of the reaction increased significantly, to 102±3°.

For the reaction of the surface oxide with HF represented in FIG. 1D to proceed, the organic liquid used (e.g., octane) must recede and expose some of the porous Si—SiO₂ layer to the HF reactant solution.

The chemical reaction between HF and the portion of the porous Si—SiO₂ film exposed by the liquid mask apparently propagates into the porous film at a rate sufficiently slow to allow temporal control. A series of samples were prepared as a function of the time of exposure of the liquid masked sample to aqueous HF.

Thermal hydrosilylation (FIG. 1E).—The surface layer of Si—H species formed by liquid mask on each of the series was then modified by hydrosilylation with neat 1-dodecene or a 10% (v/v) solution of 10-bromo-1-decene in mesitylene. The C—H stretching vibrations characteristic of the aliphatic organic chain of 1-dodecene were apparent in the FTIR spectrum at 2850 cm⁻¹ and 2925 cm⁻¹. The surface energy of each resulting dodecyl-terminated surface was quantified by sessile drop water contact angle measurements.

Using standard Schlenk and syringe inert atmosphere handling methods (Shriver, D. F.; Drezdzon, M. A., The Manipulation of Air-Sensitive Compounds. 2nd ed.; John Wiley and Sons, Inc.: New York, 1986; p 7-44), the samples were submerged in the alkene and degassed with 3 freeze-pump-thaw cycles prior to heating at 140° C. for 2 hr in a nitrogen environment. The modified samples were then rinsed with acetone and ethanol to remove excess alkene.

### Example 3

Optical measurement of the fractional filling of a partially oxidized porous Si—SiO2 film containing various inert liquids. FIG. 4 demonstrates the optical measurement of the fractional filling of a partially oxidized porous Si—SiO2 film containing the indicated organic liquids, as a function of time exposed to liquid water. A small aliquot of the organic liquid was first applied to the porous Si—SiO2 film in the optical cell. The cell was then flooded with liquid water, and a series of optical reflectance spectra were acquired in situ. Values of 2nL, obtained from the reflectance spectra were fit to a three-component Bruggeman effective medium model that included refractive index values of the porous Si—SiO2 skeleton, the organic liquid and water to determine the amount of organic liquid remaining in the pores during the course of the experiment. Data are presented as the fractional filling of the pores, defined as the fraction of the open pore volume that is filled with the organic liquid (the other fraction is assumed to be occupied by water as it infiltrates and displaces the organic layer). It is assumed that the organic and aqueous phases are completely immiscible, and that no mixing of the two liquids occurs. A fractional filling number of 1 indicates that 100% of the pore volume is filled with the organic liquid.

### Example 4

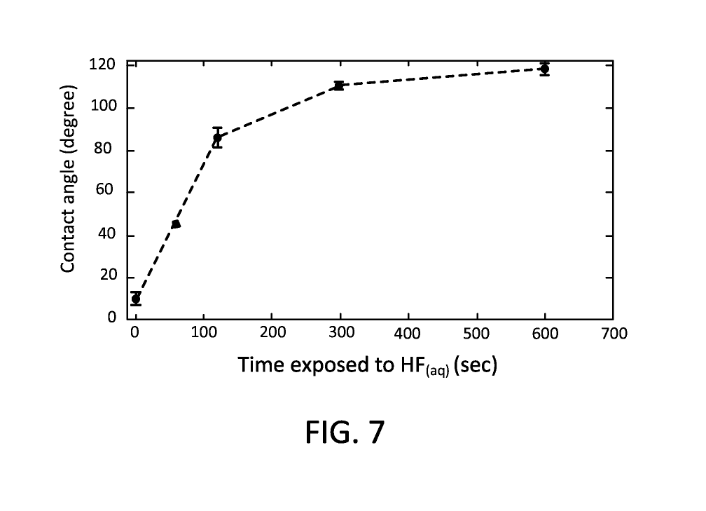

Measuring sessile drop contact angle of 1-dodecyl-modified liquid. FIG. 7 compares contact angles measured on the series of samples prepared with different times of exposure of the octane-infiltrated liquid to aqueous HF (FIG. 1D). FIG. 7 illustrates measured sessile contact angle of 1-dodecyl-modified liquid masked porous Si samples as a function of time of exposure of the octane-masked film to aqueous HF. The inert liquid (octane) masked film was exposed to 0.77% aqueous HF for the indicated time, and the resulting Si—H surface layer was subsequently modified by thermal hydrosilylation of 1-dodecene (FIG. 1E). All measurements were obtained in triplicates. The error bars shown in the graph indicate one standard deviation.

When the sample was not exposed to aqueous HF (i.e., at time point 0, FIG. 7), the porous Si—SiO₂ surface was quite hydrophilic (contact angle 10±3°). The contact angle increased significantly with HF exposure times between 0 and 300 s and then leveled off for times >300 s, indicating that the extent of the reaction that forms hydride species can be readily controlled. Extending the aqueous HF exposure time of the octane-infiltrated porous Si film to 10 min yielded, upon subsequent thermal hydrosilylation with alkene, a very hydrophobic top surface with contact angle 118±3°.

### Example 5

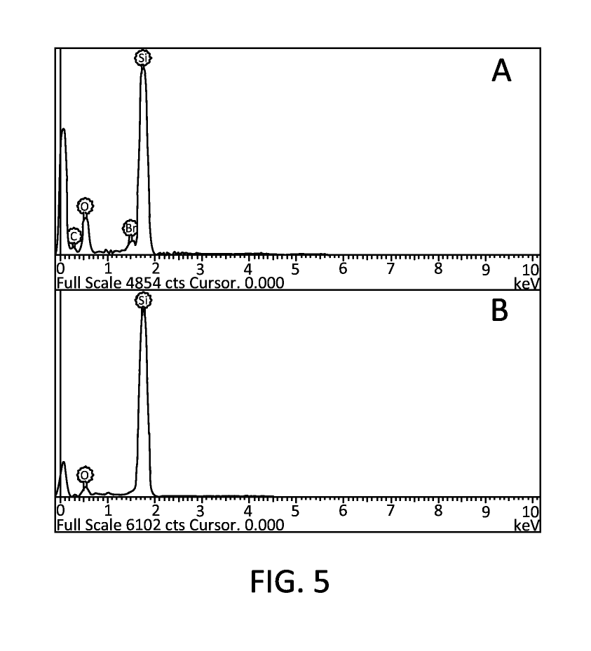

Monitoring of Liquid Transport Through Dodecyl-Modified Porous Si Samples. Despite the strong hydrophobic nature of the topmost, dodecyl-modified layer of the porous Si film, the EDX analysis (FIG. 5) showed that the lower portion of the porous Si film remained oxidized. Thus, the structures are somewhat reminiscent of an inverse micelle, consisting of a hydrophobic film coating an inner hydrophilic core. Unlike a micelle, the structures formed by liquid masking are rigid, and they can be probed by optical interferometry. The hydrophobic dodecene layer was covalently grafted to the porous Si layer, and it apparently formed a uniform, continuous coating. The transport of water across the resulting dodecyl-modified porous Si surface layer and into the underlying hydrophilic porous Si—SiO2 layer was probed using optical interferometry. A CCD-based spectrometer and white light source were coupled to the optics via a bifurcated optical fiber that allowed acquisition of optical reflectance spectra at a time resolution of 1 sec. Reflective interferometric Fourier transform spectroscopy (RIFTS) was employed, which quantified the appearance of water in the underlying porous Si layer as a shift in its optical thickness due to an increase in the average refractive index of the porous layer as air filling the pores was displaced by water.

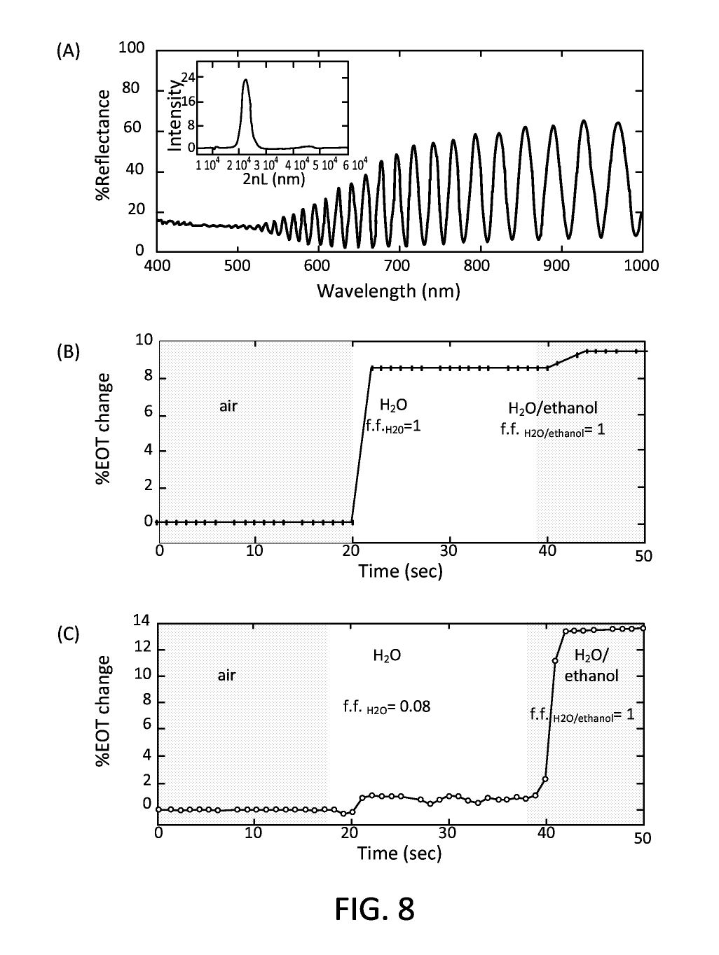

FIG. 8 illustrates experimental optical response vs time data showing the penetration of water (n_{D}=1.333) and a water/ethanol (equal volume) solution (n_{D}=1.3598) through the thin, hydrophobic dodecyl barrier layer grafted to the top portion of a partially oxidized porous Si—SiO2 film. The water infiltration was quantified by reflective interferometric Fourier transform speroscopy (RIFTS). (A) Optical reflectance spectrum of a typical sample prepared by liquid masking. Sample consists of ˜6 μm-thick surface-oxidized, hydrophilic porous Si—SiO2 layer underneath a ˜300 nm-thick dodecyl-terminated, hydrophobic layer. Inset shows the FFT of the frequency spectrum; the peak position yields the value of 2nL (the effective optical thickness) of the film. (B) The quantity 2nL measured as a function of time during water infiltration into a control sample consisting of partially oxidized porous Si, without the hydrophobic barrier layer. (C) The quantity 2nL measured as a function of time during water infiltration into a sample containing dodecyl barrier layer. Contact angle of barrier layer in this experiment was 118±3°.

The optical reflectance spectrum of a dodecyl-modified sample in air (FIG. 8A) displays Fabry-Pérot interference fringes, corresponding to constructive and destructive interference from light reflected at the air/porous Si and porous Si/crystalline Si interfaces (Hecht, E., Optics. 3rd ed.; Addison-Wesley: Reading, Mass., 1998; p 377-428). The peak maximum for each of the spectral fringes follows the Fabry-Pérot interference relationship represented by eq (1) in normal incidence:

mλ=2nL (1),

where m is the spectral order of the fringe at wavelength λ, n is the average refractive index of the porous layer and its contents, and L is the physical thickness of the film. The dodecyl-modified top portion of the film is too thin (<500 nm) to be distinguished from the underlying oxidized layer in the interference spectrum, and so the entire layer is probed as an average in this experiment. In the RIFTS method (Sailor, M. J., Porous Silicon in Practice: Preparation, Characterization, and Applications. Wiley-VCH: Weinheim, Germany, 2012; p 249), the fast Fourier transform (FFT) of the frequency spectrum (inset, FIG. 8A) yields a peak whose position along the x-axis represents the value of the effective optical thickness (EOT), or 2nL, from eq. 1.

The optical measurement conveniently monitors the infiltration of water into the porous Si—SiO2 layer in real time. The samples were mounted in a sealed cell fitted with the optical microscope/spectrometer focused on a ˜1 mm spot on the porous Si sample. The spectral data from a control experiment, performed on a porous Si—SiO2 film that had not been subjected to the process of making described herein is shown in FIG. 8A. Introduction of water to the sample chamber resulted in an instantaneous increase in the value of 2nL measured from the sample, as the liquid water replaced the air in the 47% porous film. Using the thickness of the porous Si film measured by SEM and the refractive index of air (n_{D}=1.00) and water (n_{D}=1.3330) at 20° C. (Segal, E.; Perelman, L. A.; Cunin, F.; Renzo, F. D.; Devoisselle, J.-M.; Li, Y. Y.; Sailor, M. J., Confinement of Thermoresponsive Hydrogels in Nanostructured Porous Silicon Dioxide Templates. Adv. Funct. Mater. 2007, 17, 1153-1162), a fit to the Bruggeman effective medium model (Bohren, C. F.; Huffman, D. R., Adsorption and scattering of light by small particles. Wiley: New York, 1983; p 217; Thei, W.; Henkel, S.; Arntzen, M., Connecting microscopic and macroscopic properties of porous media: choosing appropriate effective medium concepts. Thin Solid Films 1995, 255 (1-2), 177-180) was used to determine the fractional filling of the porous volume occupied by the infiltrated liquid. A fractional filling value of 1.0 was observed, indicating full infiltration of water in this sample.

The experimental protocol followed in the water infiltration experiments involved addition of a small quantity of ethanol to the sample cell several seconds after water was introduced. The purpose of the ethanol addition was twofold: (1) ethanol reduces the surface tension of water and thus allows it to more thoroughly wet the nanometer scale pores in the film; and (2) the larger refractive index of ethanol (n=1.3336) introduces a secondary increase in the value of 2nL. Both of these factors provide verification of the fraction of the porous film that has been infiltrated by water. In the case of the control sample consisting of porous Si—SiO2 with no hydrophobic barrier layer (FIG. 8B), ethanol addition resulted in an increase in 2nL that fit the calculated prediction for a fully infiltrated layer.

The presence of the thin hydrophobic layer on the top portion of the film dramatically changes its behavior with water. As the contact angle measurements demonstrate, the dodecyl-terminated layer is quite hydrophobic, and it was found to effectively exclude water from the underlying porous Si—SiO2 layer. Addition of water to the optical cell resulted in a fractional filling of only 0.08 with this sample FIG. 8C. When ethanol was added to the water, rapid penetration of the hydrophobic layer was observed, and complete infiltration of the porous Si—SiO2 layer occurred within 4 sec. In the data shown in FIG. 8C, the sample was stable, with no additional water infiltration observed, for 20 sec prior to ethanol addition. In separate experiments (not shown), it was found that pure water penetrated the hydrophobic barrier layer very slowly. It is concluded that the presence of the hydrophobic barrier layer effectively slows the penetration of water molecules into the pores.

### Example 6

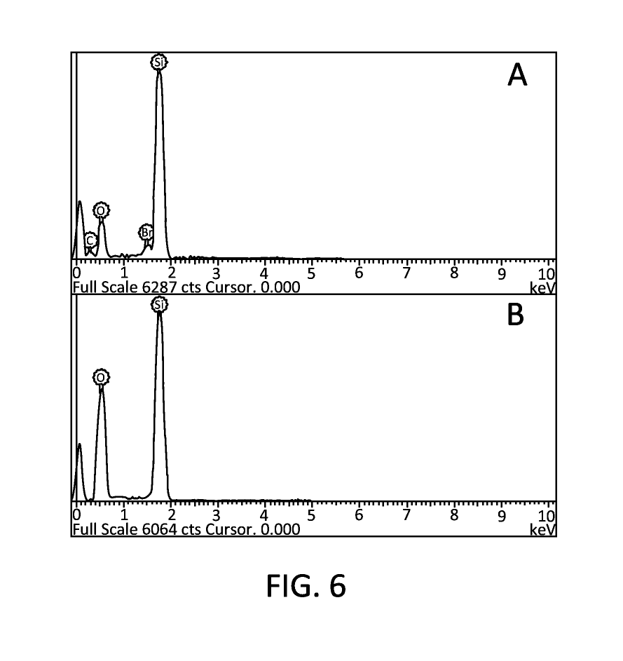

Determining the Depth of Penetration of the Chemical Reaction Front. Cross-sectional elemental mapping was used to determine the depth of penetration of the chemical reaction front, using energy dispersive X-ray spectroscopy (EDX) in the scanning electron microscope. To better resolve the depth of the reaction front, 10-bromo-1-decene was used in the hydrosilylation step instead of 1-dodecene. EDX elemental scans for Si, O, and Br were obtained from the top, middle and bottom regions of the porous Si—SiO2 film, with a resolution of ˜1 μm (See, FIG. 5). Confirmatory EDX spectra of 10-bromo-1-decyl-modified and thermally oxidized porous Si surfaces were obtained in plan view (See, FIG. 6). The EDX spectrum of the top 1 μm of porous Si from the air/porous Si interface, obtained from the cross-sectional images, revealed the presence of bromine and carbon peaks that can be attributed to grafted 10-bromo-1-decene. Bromine and carbon peaks were absent in the x-ray emission spectra obtained from the bottom portion of the porous Si layer (near the porous Si/bulk silicon interface), suggesting that the attachment of 10-bromo-1-decene preferentially occurs near the top surface. The results are consistent with the proposed liquid masking mechanism, where reactive hydride species only form in the topmost region of the porous layer, above the immiscible interface between octane and aqueous HF. The resolution of the EDX method is not sufficient to obtain accurate measurement of the thickness of 10-bromodecyl layer.

### Example 7

Loading of Rhodamine B into Modified Porous Si Films. The organic dye rhodamine B was used as a test molecule for loading into the porous Si—SiO₂ matrix. The functionalized porous Si chip was immersed in 1 mL of 0.2 mg/mL rhodamine B in acetonitrile in a glass vial and agitated for 12 h at room temperature. The sample was then removed and rinsed with acetonitrile to eliminate excess free dye not loaded into the porous reservoir. To determine the loading efficiency, the loaded dye was extracted from the porous matrix by immersion in acetonitrile for 16 hr at 37° C. with mild agitation. The quantity of rhodamine B released into solution was determined from the absorption spectrum, collected in the spectral range 400-650 nm using a SpectraMax absorbance spectrometer (Molecular Devices). The concentration of rhodamine B was determined from calibration curves of the absorbance at 552 nm and assuming Beer's law.

Dye Release Studies. Porous Si—SiO₂ chips containing loaded dye were first dried in vacuum. Samples were then immersed in 1 mL of aqueous phosphate buffered saline (PBS) solution (pH 7.4) at 37° C. with mild agitation. The supernatant containing released dye was collected every 2 hr over a 12 hr period and replaced with 1 mL of fresh buffer. Concentrations of the released rhodamine B were determined from the absorbance at 552 nm, using calibration curves of the dye in PBS.

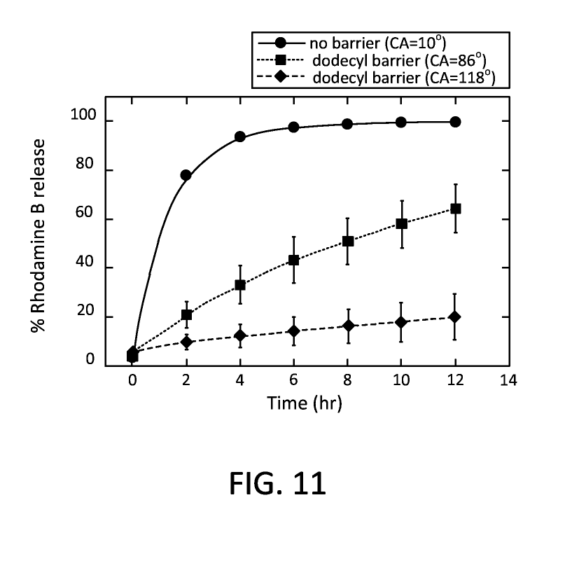

Controlled Release of Small Molecules Through the Dodecyl Barrier Layer. The ability of the hydrophobic barrier layer to impede water transport has interesting implications for controlled release drug delivery. To test the ability of water soluble molecules to escape through the dodecyl barrier layer, rhodamine B was loaded into the oxidized layer by physical adsorption from an acetonitrile solution. For comparison of transport rates, three porous Si sample preparations were tested. The procedure described in FIG. 7, where the time of exposure to HF_{(aq)} was varied in order to generate differing barrier layer thicknesses, was used to prepare two different types of dodecyl barrier layers, of contact angle 86±5° and 118±3°. The third sample type consisted of partially oxidized porous Si with no barrier layer (contact angle 10±3°). The loading efficiency for rhodamine B was 40.5±6.4 μg of dye per mg of porous Si for the barrier layer sample with 80° contact angle and 16.8±1.9 μg of dye per mg of porous Si for the sample that was 118° in contact angle. For the oxidized porous Si sample with no barrier layer, 81.6±8.9 μg of dye was loaded per mg of porous Si, representing the highest loading efficiency of the 3 surface types. After drying, the samples were immersed in a phosphate buffered saline (PBS, pH=7.4) solution and the appearance of the dye in the solution was monitored by absorbance spectroscopy for a 12 h-period (FIG. 11).

Due to the low wettability of the hydrophobic dodecyl barrier layer, transport of dye from the partially oxidized reservoir layer into aqueous solution is expected to be impeded as the aqueous medium does not easily penetrate into the pores. As shown in FIG. 11, egress of rhodamine B from the samples with no barrier layer displayed a typical burst release characteristic, with 100% of the loaded molecule released into solution within 12 h. The porous Si—SiO₂ samples with a dodecyl barrier layer exhibited significantly lower rates of release, with the rate dependent on the contact angle of the dodecyl layer. The slowest release of rhodamine was observed on most hydrophobic sample (contact angle 118°), with only 10% of the drug released in the 12 h study period.

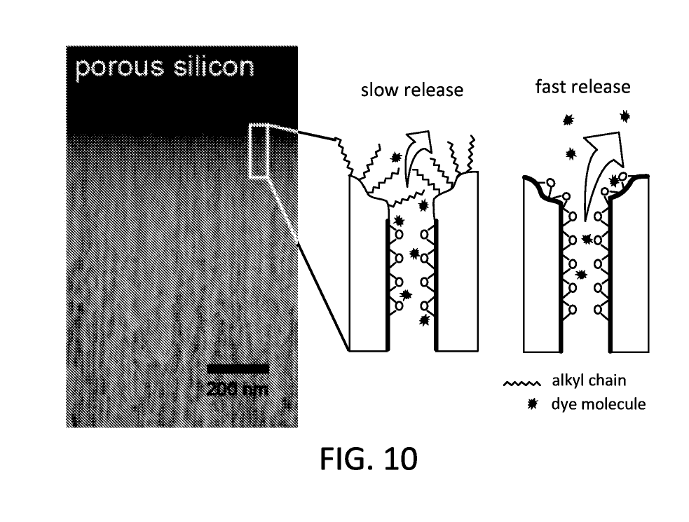

FIG. 10 illustrates that the porous silicon having a hydrophobic barrier layer at the pore mouths in porous Si allows slower transport/release of the dye molecule.

### Experimental Techniques

Scanning Electron Microscopy. An FEI XL30 ultra-high resolution scanning electron microscope (SEM) operating at an accelerating voltage of 5 kV was used to obtain plan-view and cross-sectional images of the samples. Samples were not coated with metal or carbon prior to imaging, and low beam currents were used to avoid sample charging artifacts. Energy-dispersive X-ray spectroscopy (EDX) analysis was performed on plan-view and cross-sectional samples using a Philips XL-30 Field Emission ESEM with Oxford EDX attachment.

Infrared Spectroscopy. Attenuated total reflectance Fourier transform infrared (ATR-FTIR) spectra were acquired on a Thermo Scientific Nicolet 6700 FT-IR spectrometer with a Smart iTR accessory for ATR sampling. 128 scans were averaged. Spectral resolution was 4 cm⁻¹ over the range 600-4000 cm⁻¹.

Water Contact Angle Measurement. Water contact angle measurements were obtained by imaging water droplets placed on horizontally oriented porous Si samples using a Canon EOS XSi digital camera with 100 mm macro lens. Droplets of 5 μL deionized water were placed on the sample surfaces. The contact angle was measured from the acquired images using Adobe Photoshop CS4 (Adobe Systems, Inc.) Each reported contact angle represents the average of triplicate measurements at different locations on the porous Si surfaces.

Optical Reflectance Spectra. The thin film interference spectra were obtained in a 180° reflectance configuration, collected using an Ocean Optics 4000 CCD spectrometer fitted with a bifurcated fiber optic cable. An unpolarized tungsten light source was focused onto the porous Si surface with a spot size of approximately 1 mm². Reference spectra were obtained from a broadband metallic minor (model 10D20ER.2, 25.4 mm dia front-surface silver minor on a PYREX® glass support, Newport Corporation). Optical spectra were processed using a computer and algorithms described previously. (Hecht, E. Optics. 3^{rd} ed.; Addison-Wesley: Reading, Mass., 1998; p 377-428).

Porosity and Fractional Filling Determinations by Spectroscopic Liquid Infiltration Method (SLIM). The SLIM method was used as described in the literature. Sailor, M. J., Porous Silicon in Practice: Preparation, Characterization, and Applications. Wiley-VCH: Weinheim, Germany, 2012; p 249. Briefly, two reflectance spectra of the porous Si film were obtained: (1) with the sample in air and (2) with the sample wetted with ethanol. The values of 2nL, obtained from the Fourier transform of the optical spectra, were fit to a two component Bruggeman model using the values of the refractive index of air and ethanol to determine the porosity and the thickness of the porous Si film. The thickness values determined in this fashion were validated on similar samples using cross-sectional SEM imaging. The fractional filling of water into the porous Si—SiO2 layer was calculated with a similar optical measurement and model, using the thickness and porosity values previously determined from the SLIM measurements using pure ethanol as a filling liquid. The refractive index of all liquids used were independently measured with a Mettler Toledo Refracto 30GS refractometer.

All patents, patent publications, and other published references mentioned herein are hereby incorporated by reference in their entireties as if each had been individually and specifically incorporated by reference herein.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined by reference to the appended claims, along with their full scope of equivalents.

What is claimed:

1. A silicon containing material having a plurality of pores, the material comprising: a) an exterior surface region comprising a first terminal group; and b) an interior pore surface region comprising a second terminal group, wherein the first terminal group and the second terminal group are different from each other and are chemically linked to the material.

2. The material of claim 1, wherein one of the first and second terminal groups comprises a hydride terminal group.

3. The material of claim 2, wherein the hydride terminal group comprises silicon hydride.

4. The material of claim 3, wherein the hydride terminal group is modified to an organosilane.

5. The material of claim 4, wherein the organosilane comprises an alkyl, a carboxylic acid, an ester, an amine, a protein, an oligonucleotide, a short chain peptide, a sugar, a polysaccharide, a fatty acid, or mixtures thereof.

6. The material of claim 5, wherein the organosilane comprises an alkyl.

7. The material of claim 2, wherein the other one of the first and second terminal groups comprises carbon, silicon oxide, silicon dioxide or mixtures thereof.

8. The material of claim 7, wherein the other one of the first and second terminal groups comprises silicon oxide.

9. The material of claim 1, wherein the plurality of pores have an average diameter of from about 1 nm to about 300 nm.

10. The material of claim 1, wherein the material is a film.

11. The material of claim 10, wherein the film has a thickness of from about 5 nm to 500 microns.

12. The material of claim 1, wherein the plurality of pores has an open porosity of from about 5% to about 95% based on the total volume of the material.

13. The material of claim 1, wherein the interior pore surface region further comprises a drug.

14. The material of claim 13, wherein the drug is selected from the group consisting of a small molecule, a protein, a peptide, an oligonucleotide, a nucleic acid, and mixtures thereof.

15. The material of claim 1, wherein the interior pore surface region further comprises a non-drug.

16. The material of claim 15, wherein the non-drug substance is selected from the group consisting of organic dye, inorganic complex, metal, metal oxide nanoparticle, and mixtures thereof.

17. The material of claim 16 wherein the organic dye is rhodamine B.

18. The material of claim 1, wherein the exterior surface is chemically or physically configured to affect the rate of transport of a drug or a non-drug substance on the pore surface.

19. The material of claim 1, wherein the interior pore surface is chemically or physically configured to affect the rate of transport of a drug or a non-drug substance on the pore surface.

20. A silicon containing material having a plurality of pores; the material comprising:
a) an exterior surface comprising a first terminal group comprising a hydride terminal group; and b) an interior pore surface comprising a second terminal group selected from the group consisting of silicon oxide, silicon dioxide, or mixtures thereof.

21. The material of claim 20, wherein the the second terminal group is further modified to include hydrocarbon, carboxylic acid, amine, haloalkane, aromatic hydrocarbon, thiol, peptide, carbon, or mixtures thereof.

22. The material of claim 20, wherein the hydride terminal group comprises silicon hydride.

23. The material of claim 22, wherein the hydride terminal group is modified to an organosilane.

24. The material of claim 23, wherein the organosilane comprises an alkyl, a carboxylic acid, an ester, an amine, a protein, an oligonucleotide, a short chain peptide, a sugar, a polysaccharide, a fatty acid, or mixtures thereof.

25. The material of claim 24, wherein the organosilane comprises an alkyl.

26. The material of claim 20, wherein the second terminal group comprises silicon oxide.

27. The material of claim 20, wherein the plurality of pores further contain a drug.

28. The material of claim 20, wherein the plurality of pores further contain a non-drug substance.

29. A method of treating a disease or disorder of the eye comprising injecting into the eye a silicon containing material comprising: a) an exterior surface region comprising a first terminal group; and b) an interior pore surface region comprising a second terminal group, wherein the first terminal group and the second terminal group are different from each other and are chemically linked to the material.

30. The method of claim 29, wherein one of the first and second terminal groups comprises a hydride terminal group.

31. The method of claim 30, wherein the hydride terminal group comprises silicon hydride.

32. The method of claim 31, wherein the hydride terminal group is modified to an organosilane.

33. The method of claim 32, wherein the organosilane comprises an alkyl, a carboxylic acid, an ester, an amine, a protein, an oligonucleotide, a short chain peptide, a sugar, a polysaccharide, a fatty acid, or mixtures thereof.

34. The method of claim 33, wherein the organosilane comprises an alkyl.

35. The method of claim 30, wherein the other one of the first and second terminal groups comprises silicon oxide, silicon dioxide, aluminum oxide, titanium oxide, titanium dioxide, or mixtures thereof.

36. The method of claim 35, wherein the other one of the first and second terminal groups comprises silicon oxide.

37. The method of claim 29, wherein the plurality of pores have an average diameter of from about 1 nm to about 300 nm.

38. The method of claim 29, wherein the material is a film.

39. The method of claim 38, wherein the film has a thickness of from about 1 μm to about 20 μm.

40. The method of claim 29, wherein the plurality of pores has an open porosity of from about 5% to about 95% based on the total volume of the material.

41. The method of claim 29, wherein the interior pore surface region further comprises a drug.

42. The method of claim 41, wherein the drug is selected from the group consisting of a small molecule, a protein, a peptide, an oligonucleotide, a nucleic acid, and mixtures thereof.

43. The method of claim 42, wherein the drug is a protein.

44. The method of claim 43, wherein the protein comprises ranibizumab or bevacizumab.

45. The method of claim 43, wherein the disease or disorder of the eye is selected from the group consisting of age related macular degeneration (AMD), choroidal neovascularization (CNV), uveitis, diabetic retinopathy, retinovasclar disease, retinal detachment (PVR) and glaucoma.

46. A method of preparing a silicon containing material comprising an exterior surface region comprising a first terminal group and an interior pore surface region comprising a second terminal group, wherein the first terminal group and the second terminal group are different from each other and are chemically linked to the material, the method comprising:
providing the material;
contacting the material with an inert liquid to infiltrate the interior pore surface region; and
immersing the material in a reactive liquid;
wherein the reactive liquid is immiscible or partially immiscible with the inert liquid.

47. The method of claim 46, wherein the material in the step of providing a material comprises a hydride terminal group in both the exterior surface and the pore surface.

48. The method of claim 46, wherein the inert liquid is selected from the group consisting of alkane, haloalkane, benzene derivative, fatty alcohol, and mixtures thereof.

49. The method of claim 48, wherein the inert liquid is a C₄-C₁₂ alkane.

50. The method of claim 49, wherein the inert liquid comprises octane.

51. The method of claim 46, wherein the reactive liquid is selected from the group consisting of hydrofluoric acid, oxidizing agent, and mixture thereof.

52. The method of claim 51, wherein the reactive liquid comprises hydrofluoric acid,

53. The method of claim 46, wherein the porous material is oxidized prior to the step of contacting the material with an inert liquid.

54. The method of claim 46, wherein the material is oxidized thermally.

55. The method claim 46, wherein the material is oxidized thermally at a temperature of from about 300° C. to about 1000° C.

56. The method of claim 53, wherein both the exterior and the interior pore surfaces of the material are oxidized to remove the hydride terminal group.

57. The method of claim 46, wherein the material is oxidized following the step of contacting the material with an inert liquid.

58. The method of claim 46 wherein the material is oxidized by immersing the porous material with the inert liquid infiltrated in the interior pore surface region in hydrogen peroxide.

59. The method of claim 57, wherein the exterior surface of the material is oxidized to remove the hydride terminal group to form the first terminal group.

60. The method of claim 46, further comprising the step of heating the material with a hydrosilylation agent.

61. The method of claim 60, wherein the hydrosilylation agent is selected from the group consisting of alkene, alkyne, and mixtures thereof.

62. The method of claim 61, wherein the hydrosilylation agent further comprises a functional group selected from the group consisting of carboxylic acid, ester amine, and mixtures thereof.

63. A method of preparing a silicon containing material comprising an exterior surface region comprising a first terminal group and an interior pore surface region comprising a second terminal group, wherein the first terminal group and the second terminal group are different from each other and are chemically linked to the material, the method comprising:
providing a material;
thermally oxidizing the material;
contacting the material with an inert liquid to infiltrate the interior pore surface region; and
immersing the material in a reactive liquid;
wherein the reactive liquid is immiscible or partially immiscible with the inert liquid.

64. A method of preparing a silicon containing material comprising an exterior surface region comprising a first terminal group and an interior pore surface region comprising a second terminal group, wherein the first terminal group and the second terminal group are different from each other and are chemically linked to the material, the method comprising:
providing a material;
contacting the material with an inert liquid to infiltrate the interior pore surface region;
immersing the material with the inert liquid infiltrated in the interior pore surface region in hydrogen peroxide; and
immersing the material in a reactive liquid;
wherein the reactive liquid is immiscible or partially immiscible with the inert liquid.
